(19) United States
(12) Patent Application Publication
(10) Pub. No.: US 2021/0046172 A1
(43) Pub. Date: Feb. 18, 2021

(54) BACTERIAL EXTRACELLULAR VESICLES HAVING REDUCED TOXICITY AND USE THEREOF

(71) Applicant: ROSETTA EXOSOME, Seoul (KR)

(72) Inventors: Yong Song Gho, Gyeongsangbuk-do (KR); Gyeong Yun GO, Gyeongsangbuk-do (KR); Chang Jin LEE, Daegu (KR); Jae Min LEE, Daejeon (KR); Jae Wook LEE, Gyeongsangbuk-do (KR); Nhung DINH THI HONG, Gyeongsangbuk-do (KR); Sang Soo KIM, Gyeongsangbuk-do (KR)

(21) Appl. No.: 17/026,924

(22) Filed: Sep. 21, 2020

Related U.S. Application Data
(63) Continuation-in-part of application No. PCT/KR2019/003285, filed on Mar. 21, 2019.

(30) Foreign Application Priority Data
Mar. 21, 2018 (KR) 10-2018-0032565
Mar. 21, 2019 (KR) 10-2019-0032189

(51) Int. Cl. A61K 39/108; A61K 31/704; A61K 39/00; A61K 35/74; A61P 35/00; C12N 1/20

(52) U.S. Cl. CPC A61K 39/0258; A61K 31/704; C12N 1/20; A61K 35/74; A61P 35/00; A61K 39/0005

(57) ABSTRACT

The present invention relates to bacteria-derived extracellular vesicles having reduced toxicity and the use thereof and, more specifically, to a pharmaceutical composition for treating or diagnosing diseases, a composition for delivering materials and a vaccine composition comprising bacterial extracellular vesicles having reduced toxicity, a method for preparing same, and the like. By using the bacterial extracellular vesicles having reduced toxicity of the present invention, in vivo or in vitro side effects can be reduced, efficacies can be increased, and thus the stability and efficacies of a therapeutic agent or a diagnostic agent, for various diseases including cancer, a drug carrier and/or a vaccine carrier can be enhanced. The bacterial extracellular vesicles having reduced toxicity and having loaded materials for disease treatment or vaccine, and a method for preparing same can he used for in vitro or in vivo treatments, drug carriers, vaccines or experiments.

### CROSS-REFERENCE TO RELATED APPLICATIONS

This application is a Continuation-In-Part of PCT International Application No. PCT/KR2019/003285 filed Mar. 21, 2019, which claims priority to Korean Patent Application No. KR 10-2018-0032565, filed Mar. 21, 2018 and Korean Patent Application No. KR 10-2019-0032189, filed Mar. 21, 2019, the disclosure of each of these applications is expressly incorporated herein by reference in their entirety.

### TECHNICAL FIELD

The present invention relates to bacterial extracellular vesicles with reduced toxicity and use thereof, and more specifically, to a pharmaceutical composition for treating or diagnosing diseases, a composition for delivering materials, and a vaccine composition comprising bacterial extracellular vesicles with reduced toxicity, a method for preparing the same.

### BACKGROUND ART

All cells, including Gram-negative and Gram-positive bacteria, are known to naturally secrete extracellular vesicles. Extracellular vesicles secreted from the Gram-negative bacteria are also known as outer membrane vesicles. Bacterial extracellular vesicles have sizes of 20 to 200 nm and contain various biologically active materials such as proteins, lipids, and genetic materials (DNA, RNA), etc. Extracellular vesicles secreted by Gram-negative and Gram-positive bacteria also have virulence factors such as lipopolysaccharides (LPS) and lipoteichoic acid (LTA), respectively. Bacterial extracellular vesicles are known to function as information carriers such as delivering proteins or genetic materials among the same species and cell signaling, to contribute to eliminating competitive organisms or enhancing bacterial survival, and to regulate the pathogenesis of infectious diseases caused by bacteria by delivering toxins to the host.

According to recent research results, various bacterial extracellular vesicles not only have direct therapeutic efficacy for various diseases including cancers, but can also be used as drug carriers for treating these diseases. In addition, bacterial extracellular vesicles have been used or developed clinically as vaccine carriers for preventing or treating various diseases such as meningitis. However, bacterial extracellular vesicles have limitations in clinical use as they may cause various adverse effects through systemic and local inflammation as well as inducing blood clotting.

In addition, mass production is required in order to use the bacterial extracellular vesicles as therapeutic agents for diseases, drug carriers, or vaccine carriers. It is necessary to select a strain that produces massively bacterial extracellular vesicles among various Gram-negative and Gram-positive bacterial strains, mutant and/or transformed strains for mass production of bacterial extracellular vesicles. In addition, it is required to accurately control medium composition for culturing bacteria. In case of lysogeny broth (LB), which is generally used for culturing bacteria, medium composition is not clearly defined, and the composition can be varied among batches, so extracellular vesicles isolated from bacteria cultured in LB are not identical in their components and therapeutic efficacy.

### DETAILED DESCRIPTION OF THE INVENTION

### Technical Problem

The present inventors have studied to solve the limitations of the related art that when using bacterial extracellular vesicles as agents for treating or diagnosing diseases, drug carriers, vaccine carriers or vaccine compositions, the components or therapeutic efficacy of the extracellular vesicles are not identical when isolated from bacteria cultured in LB which is commonly used. As a result, the present inventors had found that after culturing the bacteria in a chemically defined medium, the extracellular vesicles isolated therefrom are not only able to solve the problems, but also have an unexpected effect of reducing even toxicity of the extracellular vesicles, and then completed the present invention.

Therefore, an object of the present invention is to a pharmaceutical composition for treating or diagnosing diseases, a composition for delivering materials, and a vaccine composition comprising extracellular vesicles with reduced toxicity, which are isolated from bacteria cultured in a chemically defined medium, and a method for preparing the same.

The objects of the present invention are not limited to the aforementioned objects, and other objects, which are not mentioned above, will be clearly understood to those skilled in the art from the following description.

### Technical Solution

In order to achieve the object of the present invention, the present invention provides a pharmaceutical composition for treating or diagnosing diseases, comprising bacterial extracellular vesicles with reduced toxicity, wherein the bacteria are cultured in a chemically defined medium.

In yet another embodiment of the present invention, the bacteria may be transformed bacteria.

In yet another embodiment of the present invention, the bacteria may be bacteria transformed to reduce the toxicity of extracellular vesicles.

In yet another embodiment of the present invention, the bacteria may be bacteria having at least one genotype selected from the group consisting of ΔmsbB(ΔlpxM), ΔlpxA, ΔlpxB, ΔlpxC, ΔlpxD, ΔlpxH, ΔlpxK, ΔlpxL, and ΔwaaA.

In yet another embodiment of the present invention, the bacteria may be bacteria transformed to express a cell membrane fusion material.

In yet another embodiment of the present invention, the bacteria may be bacteria transformed to express at least one selected from the group consisting of a cell adhesion molecule, an antibody, a targeting protein, a cell membrane fusion protein itself, and fusion proteins thereof.

In yet another embodiment of the present invention, the chemically defined medium may include one or more carbon sources, one or more nitrogen sources, and one or more inorganic salts.

In yet another embodiment of the present invention, the chemically defined medium may be selected from the group consisting of M9 Minimal Medium, Dulbecco's Modified Eagle Medium(DMEM), Roswell Park Memorial Institute 1640(RPMI 1640) Medium, Minimum Essential Medium(MEM), MEMα, Opti-MEM, Iscove's Modified Dulbecco's Medium(IMDM), DMEM/Nutrient Mixture F-12(DMEM/F-12) Medium, McCoy's 5A Medium, Medium 199, Leibovitz's L-15 Medium, Connaught Medical Research Laboratories(CMRL) Medium, Ham's F-12K Medium, BGJb Medium, William's E Medium, Basal Medium Eagle(BME), Glasgow's MEM(GMEM), Brinster's Medium for Ovum Culture(BMOC), Fischer's Medium and MCDB 131 Medium.

In yet another embodiment of the present invention, the disease may include cancer. Specifically, the cancer may be selected from the group consisting of thyroid cancer, hepatic cancer, osteosarcoma, oral cancer, brain tumor, gall bladder cancer, colon cancer, lymphoma, bladder cancer, leukemia, small intestine cancer, tongue cancer, esophageal cancer, renal cancer, gastric cancer, breast cancer, pancreatic cancer, lung cancer, skin cancer, testicular cancer, penile cancer, prostate cancer, ovarian cancer, and cervical cancer.

In yet another embodiment of the present invention, the disease may be selected from the group consisting of hypertension, osteoporosis, irritable bowel syndrome, acute coronary syndrome, stroke, diabetes, atherosclerosis, obesity, peptic ulcer, Alzheimer's disease, emphysema, skin disease, skin infection, respiratory infection, urogenital infection, bone joint infection, central nervous system infection, and sepsis.

Furthermore, in yet another embodiment of the present invention, the pharmaceutical composition of the present invention may further comprise a drug of enhancing an anti-cancer effect.

In one embodiment of the present invention, the drug may be loaded into the bacterial extracellular vesicles.

In one embodiment of the present invention, the bacterial extracellular vesicles may have reduced toxicity compared to bacterial extracellular vesicles isolated from bacteria cultured in LB.

Furthermore, the present invention provides a composition for delivering materials for treating or diagnosing diseases comprising bacterial extracellular vesicles with reduced toxicity, loaded with a material for treating or diagnosing diseases, wherein the bacteria are cultured in a chemically defined medium.

In one embodiment of the present invention, the material for treatment or diagnosis may be selected from the group consisting of anti-cancer agents, anti-inflammatory agents, angiogenesis inhibitors, peptides, proteins, vaccines, toxins, nucleic acids, beads, microparticles, nanoparticles, fluorescent proteins, and quantum dots.

In another embodiment of the present invention, the nucleic acid may be selected from the group consisting of DNAs, RNAs, aptamers, locked nucleic acids (LNA), peptide nucleic acids (PNA), and morpholinos.

In yet another embodiment of the present invention, the nanoparticles may be selected from the group consisting of iron oxide, gold, carbon nanotubes, and magnetic beads.

Furthermore, the present invention provides a vaccine composition for preventing or treating diseases comprising bacterial extracellular vesicles with reduced toxicity, wherein the bacteria are cultured in a chemically defined medium.

The term “vaccine composition” used in the present invention includes one or more antigens or immunogens in a pharmaceutically acceptable carrier useful for inducing an immune response in a host. The vaccine composition of the present invention comprises bacterial extracellular vesicles with reduced toxicity, and may comprise additional antigens or immunogens.

In one embodiment of the present invention, the disease may be an infection caused by bacteria, viruses or fungi. The infection may be selected from the group consisting of a skin infection, a respiratory infection, a urogenital infection, a bone joint infection, a central nervous system infection, and sepsis.

In yet another embodiment of the present invention, the disease may be selected from the group consisting of thyroid cancer, hepatic cancer, osteosarcoma, oral cancer, brain tumor, gall bladder cancer, colon cancer, lymphoma, bladder cancer, leukemia, small intestine cancer, tongue cancer, esophageal cancer, renal cancer, gastric cancer, breast cancer, pancreatic cancer, lung cancer, skin cancer, testicular cancer, penile cancer, prostate cancer, ovarian cancer, and cervical cancer.

In yet another embodiment of the present invention, the disease may be selected from the group consisting of hypertension, osteoporosis, irritable bowel syndrome, acute coronary syndrome, stroke, diabetes, atherosclerosis, obesity, peptic ulcer, Alzheimer's disease, emphysema, and skin diseases.

In yet another embodiment of the present invention, the vaccine may be used by co-administering a drug or an immunoadjuvant for increasing efficacy or decreasing adverse effects.

In yet another embodiment of the present invention, the bacteria may be transformed bacteria.

In yet another embodiment of the present invention, the bacteria are bacteria transformed to express a fusion protein of a membrane protein of the extracellular vesicles and an antigen; or bacteria transformed to express a fusion protein of a luminal cargo of the extracellular vesicles and an antigen.

In yet another embodiment of the present invention, the antigen may be a bacterial antigen, a viral antigen, a fungal antigen, and a cancer-derived antigen; or mutants such as Ras protein, Raf protein, Src protein, Myc protein, epidermal growth factor receptor (EGFR), platelet-derived growth factor receptor (PDGFR), vascular endothelial growth factor receptor (VEGFR), p53, phosphatase and tensin homolog (PTEN), or HER2/neu.

In yet another embodiment of the present invention, an antigen protein or a peptide may be loaded into the bacterial extracellular vesicles.

In yet another embodiment of the present invention, the antigen may be a bacterial antigen, a viral antigen, a fungal antigen, and a cancer-derived antigen; or mutants such as Ras protein, Raf protein, Src protein, Myc protein, epidermal growth factor receptor (EGFR), platelet-derived growth factor receptor (PDGFR), vascular endothelial growth factor receptor (VEGFR), p53, phosphatase and tensin homolog (PTEN), or HER2/neu.

Furthermore, the present invention provides a method of reducing toxicity of bacterial extracellular vesicles, comprising (a) culturing bacteria in a chemically defined medium; and (b) isolating the bacterial extracellular vesicles secreted from the culture medium.

In yet another embodiment of the present invention, the bacteria may be transformed bacteria.

In yet another embodiment of the present invention, the bacteria may be bacteria transformed to reduce the toxicity of the extracellular vesicles.

In yet another embodiment of the present invention, the bacteria may be bacteria with a modified endotoxin producing gene.

In yet another embodiment of the present invention, the bacteria may be bacteria transformed to express a cell membrane fusion material.

In yet another embodiment of the present invention, the bacteria may be bacteria transformed to target specific cells or tissues.

In yet another embodiment of the present invention, the bacteria may be bacteria transformed to express at least one selected from the group consisting of a cell adhesion molecule, an antibody, a targeting protein, a cell membrane fusion protein itself, and fusion proteins thereof.

In yet another embodiment of the present invention, the isolating step may be performed by using a method selected from the group consisting of ultracentrifugation, density gradient, filtration, dialysis, precipitation, chromatography, and free flow electrophoresis.

Another aspect of the present invention provides a method for reducing toxicity of bacterial extracellular vesicles for delivering materials, comprising: (a) culturing bacteria in a chemically defined medium; (b) isolating bacterial extracellular vesicles secreted from the culture medium; (c) adding and culturing a material for treating or diagnosing diseases to a suspension containing the isolated bacterial extracellular vesicles; and (d) isolating the bacterial extracellular vesicles loaded with the material for treating or diagnosing diseases secreted from the culture medium.

In one embodiment of the present invention, the material for treatment or diagnosis may be selected from the group consisting of anti-cancer agents, anti-inflammatory agents, angiogenesis inhibitors, peptides, proteins, vaccines, toxins, nucleic acids, beads, microparticles, nanoparticles, fluorescent proteins, and quantum dots.

In yet another embodiment of the present invention, the nucleic acid may be selected from the group consisting of DNAs, RNAs, aptamers, locked nucleic acids (LNA), peptide nucleic acids (PNA), and morpholinos.

In yet another embodiment of the present invention, the nanoparticles may be selected from the group consisting of iron oxide, gold, carbon nanotubes, and magnetic beads.

### Advantageous Effects

By using bacterial extracellular vesicles with reduced toxicity of the present invention, it is possible to reduce adverse effects in vitro as well as in vivo, and increase efficacy, thereby enhance stability and efficacy of bacterial extracellular vesicles as agents for treating/diagnosing various diseases including cancers, and for delivering drugs and/or vaccines. Furthermore, bacterial extracellular vesicles with reduced toxicity of the present invention, which are loaded with materials for treating diseases or for vaccination, and a method for preparing the same can be used for treating diseases, drug delivery, vaccination, or experiments in vitro or in vivo.

### BRIEF DESCRIPTION OF THE DRAWINGS

FIGS. 1A-B illustrates results of showing growth curves of E. coli ΔmsbB-prsA-EGF (E. coli in which E. coli ΔmsbB with reduced toxicity of lipopolysaccharides is transformed with pHCE-prsA-EGF vector expressing a fusion protein of human EGF and bacterial inner membrane protein, PrsA) in various chemically defined media (FIG. 1A) and mammalian cell culture media (FIG. 1B).

FIGS. 2A-D illustrate results of showing growth curves of E. coli ΔmsbB, Staphylococcus aureus, Salmonella enterica, and Bacillus subtilis in low phosphate M9 medium containing vitamins and trace elements.

FIGS. 3A-B illustrates results of analyzing E. coli extracellular vesicles (FIG. 3A) and other bacterial extracellular vesicles (FIG. 3B) with dynamic light scattering.

FIGS. 4A-C illustrates results of measuring a protein amount in bacterial extracellular vesicles derived from 1 L of culture medium (FIG. 4A), the number of bacterial extracellular vesicles corresponding to 1 μg of total protein amounts (FIG. 4B), and the number of bacterial extracellular vesicles derived from 1 L of culture medium (FIG. 4C).

FIGS. 5A-B illustrates results of analyzing protein compositions present in bacterial extracellular vesicles EV^{LB}, EV^{M9}, and EV^{M9+} obtained by culturing E. coli ΔmsbB in LB, low phosphate M9 medium, and low phosphate M9 medium containing vitamins and trace elements as well as bacterial extracellular vesicles ^{EGF}EV^{LB}, ^{EGF}EV^{M9}, and ^{EGF}EV^{M9+} obtained by culturing E. coli ΔmsbB prsA-EGF in LB medium, low phosphate M9 medium, and low phosphate M9 medium containing vitamins and trace elements through SDS-PAGE (FIG. 5A) and analyzing amounts of OmpA, an outer membrane protein, through Western blotting (FIG. 5B).

FIGS. 6A-C illustrates results of verifying anti-tumor activities of bacterial extracellular vesicles in a mouse tumor model: ^{EGF}EV^{M9} obtained by culturing E. coli ΔmsbB-prsA-EGF in low phosphate M9 medium (FIG. 6A), bacterial extracellular vesicles ^{EGF}Ev^{DMEM} obtained by culturing E. coli ΔmsbB-prsA-EGF in DMEM (FIG. 6B), and bacterial extracellular vesicles EV^{LB} and ^{EGF}EV^{LB} obtained by culturing E. coli ΔmsbB and E. coli ΔmsbB-prsA-EGF in LB (FIG. 6C).

FIG. 7 illustrates a result of verifying anti-tumor activities of bacterial extracellular vesicles in a mouse tumor model: SA EV^{M9+} obtained by culturing S. aureus in low phosphate M9 medium containing vitamins and trace elements.

FIGS. 8A-B illustrate a result of quantification of doxorubicin loaded into bacterial extracellular vesicles.

FIGS. 9A-B illustrate a result of evaluating drug delivery efficacy of ^{Dox}EV^{M9+}, bacterial extracellular vesicles obtained by culturing E. coli ΔmsbB in low phosphate M9 medium containing vitamins and trace elements then loaded with doxorubicin, to colorectal cancer cells (CT26; FIG. 9A) and endothelial cells (HMEC-1; FIG. 9B). In the right panel, the concentration of ^{Dox}EV^{M9+} is 1×10¹⁰ EVs/mL, and 1 μg/mL of doxorubicin is loaded.

FIGS. 10A-B illustrates a result of evaluating drug delivery efficacy of SA ^{Dox}EV^{M9+}, bacterial extracellular vesicles obtained by culturing S. aureus in low phosphate M9 medium containing vitamins and trace elements then loaded with doxorubicin, to colorectal cancer cells (CT26; FIG. 10A) and endothelial cells (HMEC-1; FIG. 10B). In the right panel, the concentration of SA ^{Dox}EV^{M9+} is 1×10 ¹⁰ EVs/mL, and 1 μg/mL of doxorubicin is loaded.

FIGS. 11A-B illustrates a result of evaluating antibody formation efficacy of EV^{M9+} (FIG. 11A), and SA EV^{M9+} (FIG. 11B), bacterial extracellular vesicles obtained by culturing E. coli ΔmsbB and S. aureus in low phosphate M9 medium containing vitamins and trace elements, respectively, directed against E. coli and S. aureus extracellular vesicles, respectively.

### MODE FOR CARRYING OUT INVENTION

The present invention provides a pharmaceutical composition for treating or diagnosing diseases, comprising bacterial extracellular vesicles with reduced toxicity cultured in a chemically defined medium.

Bacteria

In the present invention, the bacteria include Gram-negative or Gram-positive bacteria. The Gram-negative bacteria include E. coli, Pseudomonas aeruginosa, S. enterica, etc., and the Gram-positive bacteria include S. aureus and B. subtilis, but are not limited thereto.

The bacteria of the present invention include transformed bacteria. Specifically, the transformed bacteria include bacteria transformed to reduce toxicity of the extracellular vesicles, for example, endotoxin-producing genetically modified bacteria, specifically E. coli ΔmsbB, but are not limited thereto. Furthermore, the bacteria also include bacteria transformed to target specific cells or tissues, for example, bacteria transformed to target tumor vasculatures, tumor tissues, and tumor cells. In addition, the bacteria used in the present invention include bacteria transformed to be fused with a cell membrane of a target cell, bacteria transformed to express a material for treating and/or diagnosing diseases, and bacteria transformed to inhibit a specific material and express the specific material at the same time. However, the bacteria for preparing the bacterial extracellular vesicles of the present invention are not limited thereto.

The bacteria may be transformed by treating materials or introducing genes, and may be transformed two or more times.

In one embodiment of the present invention, the bacteria may be transformed to inhibit expression of one or more specific proteins.

In one embodiment of the present invention, the bacteria may be transformed to express at least one selected from the group consisting of a cell adhesion molecule, an antibody, a targeting protein, a cell membrane fusion protein, or fusion proteins thereof.

In one embodiment of the present invention, the bacteria may include E. coli ΔmsbB-prsA-EGF obtained by transforming E. coli ΔmsbB with reduced toxicity of lipopolysaccharides with pHCE-prsA-EGF vectors expressing a fusion protein of a human epidermal growth factor (EGF) and a bacterial inner membrane protein PrsA, but are not limited thereto.

Chemically Defined Medium

The ‘chemically defined medium’ of the present invention is in contrast to a ‘natural medium’ using a natural material having an unclear composition such as serum, tissue extract, etc., and refers to a chemically defined medium prepared by only a material with clear components and chemical properties of the composition. The ‘chemically defined medium’ may also be expressed as a ‘chemically-defined medium’, that is, may be defined as a medium suitable for culture of eukaryotic cells, bacteria, etc., in which the composition and content of chemical components contained in the medium have been identified. As described above, the chemically defined medium is a required component for solving the problems caused by LB or nutrient broth, which has been widely used for the preparation of bacterial extracellular vesicles in the related art, and producing extracellular vesicles exhibiting a uniform effect.

In one embodiment of the present invention, the chemically defined medium may include one or more carbon sources, one or more nitrogen sources, and one or more inorganic salts. The types and contents of the carbon source, the nitrogen source, and the inorganic salt included in the chemically defined medium are not particularly limited, and may be appropriately adjusted by those skilled in the art according to the properties of the bacteria to be cultured and culture conditions.

In one non-limiting embodiment of the present invention, the carbon source may be glucose, glycerol, fructose, lactose, sucrose, arabinose, or a mixture thereof, the nitrogen source may be an ammonium salt, ammonium hydroxide, ammonium ions, amino acids or a mixture thereof, and the inorganic salt may be a sodium salt, a potassium salt, a magnesium salt, a calcium salt, a phosphate salt, or a sulfate salt.

In one embodiment of the present invention, the chemically defined medium may further include components used to incubate one or more eukaryotic cells or bacteria to be listed below:

(1) salts (e.g., sodium, potassium, magnesium, calcium, etc.) that contribute to the osmolality of the medium; (2) essential amino acids, (3) vitamins and/or other organic compounds required in low concentrations; and (4) trace elements, wherein the trace elements may be typically defined as inorganic compounds required at very low concentrations, usually in a micromolar range; (4) buffers, antioxidants, stabilizers against mechanical stress, or proteases; (5) other nutritionally required supplements, including (a) animal serum; (b) hormones and other growth factors such as insulin, transferrin, and epidermal growth factors; and (c) hydrolysates of plants, yeast and/or tissues, including protein hydrolysates.

In one embodiment of the present invention, the vitamin may include D-biotin, choline chloride, folic acid, myoinositol, niacinamide, pyridoxine HCl, D-pantothenic acid (hemiCa), riboflavin, thiamine HCl, vitamin B12 or a mixture thereof.

In one embodiment of the present invention, the chemically defined medium may be a commercially available basic medium or a medium in which the above-described components are added to the basic medium. Non-limiting examples of the commercially available basic medium may include M9 Minimal Medium, Dulbecco's Modified Eagle Medium (DMEM), Roswell Park Memorial Institute 1640 (RPMI 1640) Medium, Minimum Essential Medium (MEM), MEMα, Opti-MEM, Iscove's Modified Dulbecco's Medium (IMDM), DMEM/Nutrient Mixture F-12(DMEM/F-12) Medium, McCoy's 5A Medium, Medium 199, Leibovitz's L-15 Medium, Connaught Medical Research Laboratories (CMRL) Medium, Ham's F-12K Medium, BGJb Medium, William's E Medium, Basal Medium Eagle (BME), Glasgow's MEM (GMEM), Brinster's Medium for Ovum Culture (BMOC), Fischer' s Medium and MCDB 131 Medium, but are not limited thereto.

Disease to be Treated or Diagnosed

The ‘disease’ of the present invention may include various diseases including cancer. In one embodiment of the present invention, the cancer may be selected from the group consisting of thyroid cancer, hepatic cancer, osteosarcoma, oral cancer, brain tumor, gall bladder cancer, colon cancer, lymphoma, bladder cancer, leukemia, small intestine cancer, tongue cancer, esophageal cancer, renal cancer, gastric cancer, breast cancer, pancreatic cancer, lung cancer, skin cancer, testicular cancer, penile cancer, prostate cancer, ovarian cancer, and cervical cancer, but is not limited thereto.

In another embodiment of the present invention, the disease may be selected from the group consisting of hypertension, osteoporosis, irritable bowel syndrome, acute coronary syndrome, stroke, diabetes, atherosclerosis, obesity, peptic ulcer, Alzheimer's disease, emphysema, skin disease, skin infection, respiratory infection, urogenital infection, bone joint infection, central nervous system infection, and sepsis, but is not limited thereto.

Bacterial Extracellular Vesicles

The ‘bacterial extracellular vesicles’ of the present invention include ‘shedding extracellular vesicles’ naturally secreted from bacteria, and ‘artificial extracellular vesicles’ artificially prepared from bacteria using genetic, chemical, or mechanical methods.

The ‘bacterial extracellular vesicle’ of the present invention has the inside and the outside divided by a lipid bilayered membrane made of a cell membrane component of the derived bacteria, and has a plasma membrane lipid, a plasma membrane protein, nucleic acid, bacterial components, and the like, which means that the size is smaller than that of the original bacteria, but is not limited thereto.

The bacterial extracellular vesicles of the present invention may be obtained by various methods, and examples thereof are introduced as following, but the present invention is not limited thereto.

(1) Bacteria or transformed bacteria are cultured, and the culture medium is filtered and ultra-centrifuged to obtain shedding extracellular vesicles.

(2) Bacteria or transformed bacteria are treated with a detergent, and the culture medium is filtered and ultra-centrifuged to obtain shedding extracellular vesicles. The detergent is not limited.

(3) Bacteria or transformed bacteria are treated with an antibiotic, and the culture medium is filtered and ultra-centrifuged to obtain shedding extracellular vesicles. The antibiotic is not limited, and includes gentamicin, ampicillin, kanamycin, and the like.

The artificial extracellular vesicles of the present invention may be prepared by using a method selected from the group consisting of extrusion, sonication, cell lysis, homogenization, freeze-thaw, electroporation, mechanical degradation, and chemical treatment of a suspension containing bacteria, but are not limited thereto.

In one embodiment of the present invention, the bacterial extracellular vesicles cultured in the chemically defined medium may be characterized by having reduced toxicity, and more specifically, characterized by having reduced toxicity compared with bacterial extracellular vesicles cultured in LB.

In one embodiment of the present invention, the bacterial extracellular vesicles cultured in the chemically defined medium may be characterized by having unchanged pharmacological activity of the extracellular vesicles themselves, but having reduced toxicity, and more specifically, characterized by having no large differences in pharmacological activity, but having reduced toxicity compared to bacteria-derived extracellular vesicles cultured in LB.

In one embodiment of the present invention, the bacterial extracellular vesicles cultured in the chemically defined medium may be characterized by having increased pharmacological activity of the extracellular vesicles themselves and reduced toxicity, and more specifically, characterized by having increased pharmacological activity, but having reduced toxicity compared to bacterial extracellular vesicles cultured in LB.

In one embodiment of the present invention, the pharmacological activity of the extracellular vesicles may be anti-tumor activity.

In one embodiment of the present invention, the membrane of the bacterial extracellular vesicles may further include components other than the cell membrane of the bacteria.

The components other than the cell membrane may include a targeting material, a cell membrane fusion material (fusogen), cyclodextrin, polyethylene glycol, and the like. In addition, the components other than the cell membrane may be added by various methods, and include chemical modification of the cell membrane, and the like.

For example, the membrane components of the bacterial extracellular vesicles may be modified by a chemical method using a thiol group (—SH) or an amine group (—NH₂), or the membrane components of the bacterial extracellular vesicles may be chemically modified by chemically binding polyethylene glycol to the bacterial extracellular vesicles.

In the preparation of the bacterial extracellular vesicles of the present invention, the chemically modification of the membrane components of the bacterial extracellular vesicles may be further included.

Pharmaceutical Composition for Treating or Diagnosing Disease

In one embodiment of the present invention, the pharmaceutical composition may further comprise a drug of inhibiting the toxicity by the extracellular vesicles. In addition, the drug may be loaded into the extracellular vesicles. The drug includes a drug of inhibiting the toxicity caused by endotoxin, for example, may include polymyxin B.

In yet another embodiment of the present invention, the pharmaceutical composition may further comprise a drug of enhancing an anti-tumor activity. In addition, the drug may be loaded into the extracellular vesicles.

In the present invention, ‘loading’ refers to exposing a required material to the surface of the bacterial extracellular vesicles or encapsulating the material therein, but is not limited thereto.

In the present invention, the drug of increasing the anti-tumor activity includes a drug inhibiting an immune response of T helper 17 cells (Th17), a drug inhibiting the production or activity of interleukin (IL)-6, a drug inhibiting the production or activity of a vascular endothelial growth factor (VEGF), a drug inhibiting signaling of signal transducer and activator of transcription 3 (STAT3), an anti-cancer agent, a drug-loaded nanoparticle therapeutic agent, a cell therapeutic agent for cancer treatment, and the like. An example of the drug inhibiting the Th17 immune response may include aspirin, and an example of the drug inhibiting the production or activity of the VEGF may include a drug inhibiting signaling by a VEGF receptor. An example of a liposome loaded with an anti-cancer agent may include DOXIL. The nanoparticle therapeutic agent may include a liposome, a dendrimer, a polymer, an extracellular vesicle, etc., as a particle having a size of 10 nm to 10 μm, but is not limited thereto.

The pharmaceutical composition in the present invention may further include a pharmaceutically acceptable carrier in addition to the active ingredients. That is, saline, sterile water, a Ringer's solution, buffered saline, cyclodextrin, a dextrose solution, a maltodextrin solution, glycerol, ethanol, liposome, and at least one of these ingredients may be mixed and used, and other general additives such as antioxidants and buffers may be further included if necessary. In addition, the pharmaceutical composition may be formulated to injectable formulations such as an aqueous solution, a suspension, an emulsion and the like, pills, capsules, granules, or tablets by additionally adding a diluent, a dispersant, a surfactant, a binder, and/or a lubricant.

Method for Treating and/or Diagnosing Cancer

Yet another aspect of the present invention provides a method for treating and/or diagnosing cancer comprising administering the bacterial extracellular vesicles to a subject.

In the present invention, the term ‘subject’ refers to a subject in need of treatment for a specific disease (for example, cancer, vascular disease, or inflammatory disease), and more specifically, refers to human or non-human primates, mammals, such as mice, rats, dogs, cats, horses and cattle, fish and birds.

In the present invention, ‘cancer’ refers to a disease group having a characteristic of over-proliferating cells and penetrating into surrounding tissues when the normal apoptosis balance is broken. A target to be treated in the present invention may be selected from the group consisting of carcinoma derived from epithelial cells, such as lung cancer, laryngeal cancer, gastric cancer, colorectal cancer, hepatic cancer, gall bladder cancer, pancreatic cancer, breast cancer, cervical cancer, prostate cancer, renal cancer, and skin cancer, sarcoma derived from connective tissue cells, such as bone cancer, muscle cancer, fat cancer, and fibroblast cancer, blood cancer derived from hematopoietic cells, such as leukemia, lymphoma, and multiple myeloma, tumors caused in the nervous tissue, and the like, but is not limited thereto.

The bacteria and the bacterial extracellular vesicles used in the method of the present invention are as described above.

In one embodiment of the present invention, the method may use bacterial extracellular vesicles loaded with a drug in order to reduce adverse effects of the extracellular vesicles. The drug may be a drug (e.g., polymyxin B) that inhibits the activity of endotoxin, and may be a drug (e.g., aspirin) that has anti-inflammatory and/or anticoagulant activity. The bacterial extracellular vesicles and aspirin can be co-administered to prevent adverse effects such as inflammatory responses and blood coagulation caused by the bacterial extracellular vesicles. In addition, during culture, the extracellular vesicles may be prepared from bacteria treated with the drug.

In order to reduce the adverse effects of the bacterial extracellular vesicles, the membrane components of the extracellular vesicles may be chemically modified and used. For example, the membrane components of the extracellular vesicles may be modified by a chemical method using a thiol group or an amine group, or may be used by binding polyethylene glycol to the extracellular vesicles by a chemical method.

In yet another embodiment of the present invention, extracellular vesicles loaded with a drug that increases anti-tumor activity may be used. The drug that increases anti-cancer efficacy is as described above.

In addition, in an embodiment of the method, when the extracellular vesicles are administered to a subject, a drug that reduces adverse effects of the extracellular vesicles and/or a drug that increases anti-tumor activity, a drug-loaded nanoparticle therapeutic agent, a cell therapeutic agent, etc. may be co-administered.

Composition for Delivering Materials, Material Delivery Method, and Drug Delivery System

Still another aspect of the present invention provides a composition for delivering materials for treating and/or diagnosing diseases comprising bacterial extracellular vesicles loaded with the material for treating and/or diagnosing diseases.

The material to be loaded into the bacterial extracellular vesicles of the present invention is not particularly limited, and may be, for example, a material for treatment and/or diagnosis, and a material expressed by the bacteria or transformed bacteria may also be loaded, and if necessary, a material which is not derived from the bacteria, but prepared outside the bacteria may also be loaded, but is not limited thereto. That is, the materials for treatment and/or diagnosis include those derived from the bacteria and those injected from outside the bacteria. The number of materials to be loaded may also be one or two or more. In addition, the materials may be loaded into the surface of the bacterial extracellular vesicles by physical, chemical, and/or biological methods, but is not limited thereto.

The method of loading various materials for treatment and/or diagnosis into the bacterial extracellular vesicles of the present invention may use various known methods, and representatively, one of the following methods may be selected, but is not limited thereto.

First, the extracellular vesicles are prepared from bacteria that have already been loaded with materials for treatment and/or diagnosis. For example, when bacteria are cultured in a culture medium including various materials for treatment and/or diagnosis, the bacteria loaded with the material may be obtained, or the materials may also be loaded into the bacteria through an electroporation method. In addition, the material is loaded into the shedding extracellular vesicles naturally secreted from these bacteria, or artificial extracellular vesicles prepared by methods such as ultrasonication, extrusion, and mechanical degradation.

Second, in the process of preparing the bacterial extracellular vesicles, the material is loaded into the bacterial extracellular vesicles. For example, when the material is added to a solution containing bacteria and then the extracellular vesicles are prepared by extrusion through a filter having a size smaller than that of bacteria, the material is loaded into the extracellular vesicles.

Third, the material may be loaded after preparing the shedding extracellular vesicles or the artificial extracellular vesicles. For example, the material may be loaded into the shedding extracellular vesicles or the artificial extracellular vesicles, which were already prepared, by the electroporation method.

The material for treatment and/or diagnosis used in the present invention may be at least one selected from the group consisting of anti-cancer agents, anti-inflammatory agents, angiogenesis inhibitors, peptides, proteins, vaccines, toxins, nucleic acids, beads, microparticles and nanoparticles, but is not limited thereto.

The anti-cancer agent that may be used in the present invention may include DNA alkylating agents such as mechloethamine, chlorambucil, phenylalanine, mustard, cyclophosphamide, ifosfamide, carmustine (BCNU), lomustine (CCNU), streptozotocin, busulfan, thiotepa, cisplatin, carboplatin, etc. In addition, the anti-cancer agent may include anti-cancer antibiotics such as dactinomycin (actinomycin D), doxorubicin (adriamycin), epirubicin, idarubicin, mitoxantrone, plicamycin, mitomycin, and C-bleomycin. In addition, the anti-cancer agent may be selected from the group consisting of plant alkyloids such as vincristine, vinblastine, paclitaxel, and docetaxel, as well as daunorubicin, prednisone, cisplatin, herceptin, rituximab, etoposide, teniposide, topotecan, and iridotecan. In addition, radioactive materials commonly used in the art may also be used as the anti-cancer agent.

The anti-inflammatory agent that may be used in the present invention may be selected from the group consisting of dexamethasone, indomethacin, ibuprofen, clobetasol propionate, diflorasone diacetate, halobetasol propionate, amcinonide, fluocinonide, mometasone furoate, deoxymetasone, diclofenac, piroxicam, etc., but is not limited thereto.

The angiogenesis inhibitor that may be used in the present invention includes all known drugs to be used to inhibit a process of generating new blood vessels in existing blood vessels, and the kind thereof is not particularly limited.

Examples of the protein or peptide that may be used in the present invention include growth factors such as VEGF and EGF, cytokines such as IL-1, IFN-gamma, and IL-10, various antibody therapeutic agents, etc. as well as RNase A and DNase. In addition, various proteins and peptides capable of inhibiting the growth and metastasis of cancer and inhibiting the inflammatory response may be used without limitations.

The vaccine that may be used in the present invention is to activate an immune system of the human body by administering an artificially attenuated pathogen (antigen) into the human body to prevent the infection of the pathogen, and may include the bacterial extracellular vesicles with reduced toxicity of the present invention, but is not limited thereto.

The toxin that may be used in the present invention is a generic term that is derived from various organisms and can exhibit toxicity when absorbed into the body, and may induce cell death through the toxin. The type of toxin that may be used as a therapeutic material of the present invention is not particularly limited.

The nucleic acid that may be used in the present invention may be selected from the group consisting of DNA, RNA, aptamer, locked nucleic acid (LNA), peptide nucleic acid (PNA), and morpholino, but is not limited thereto. Such nucleic acids may be used for purposes such as a sense effect, an antisense effect, RNA interference, and function inhibition of proteins.

The nanoparticles of the present invention may be nanoparticles including iron oxide, gold, carbon nanotubes, or magnetic beads, but are not limited thereto. Beads such as magnetic beads may be loaded and used into the extracellular vesicles. Magnetic particles such as iron oxide may be used as a contrast medium to obtain magnetic resonance imaging (MRI). Nucleic acids bound to nanoparticles, proteins bound to nanoparticles, etc. may also be used, and radioactive materials useful for diagnosis may also be used.

In one embodiment of the present invention, the material for treatment and/or diagnosis may be a material of emitting fluorescence, but is not limited thereto. For example, the material of emitting fluorescence may be a fluorescent protein or a quantum dot (Qdot).

In the present invention, a nucleic acid encoding a fluorescent protein or extracellular vesicles loaded with various fluorescent materials may be used for diagnosis. Fluorescence emitting quantum dots that induce cell death may also be used for therapy.

Two or more materials may be delivered using the extracellular vesicles according to the present invention. For example, two or more materials may be delivered using extracellular vesicles loaded with two or more materials simultaneously. Alternatively, two or more materials may be delivered using two or more types of extracellular vesicles loaded with one or at least two materials.

Still another aspect of the present invention provides a method for delivering a drug for treating and/or diagnosing diseases, a nanoparticle therapeutic agent loaded with a drug, and a cell therapeutic agent, characterized by using bacterial extracellular vesicles loaded with the drug for treating and/or diagnosing diseases.

Still yet another aspect of the present invention provides a drug delivery system for diagnosing and/or treating diseases using bacterial extracellular vesicles loaded with the drug for treating and/or diagnosing diseases.

Vaccine Composition

Yet another aspect of the present invention provides a vaccine composition for preventing or treating diseases comprising bacterial extracellular vesicles with reduced toxicity.

The bacteria and the bacterial extracellular vesicles of the present invention are as described above.

In one embodiment of the present invention, the disease may include infections caused by bacteria, viruses or fungi.

In another embodiment of the present invention, the infections by the bacteria include infections by Gram-negative bacteria and infections by Gram-positive bacteria. Specifically, the infection may be a skin infection, a respiratory infection, a urogenital infection, a bone joint infection, a central nervous system infection, and sepsis, but is not limited thereto.

In yet another embodiment of the present invention, the disease may be selected from the group consisting of thyroid cancer, hepatic cancer, osteosarcoma, oral cancer, brain tumor, gall bladder cancer, colon cancer, lymphoma, bladder cancer, leukemia, small intestine cancer, tongue cancer, esophageal cancer, renal cancer, gastric cancer, breast cancer, pancreatic cancer, lung cancer, skin cancer, testicular cancer, penile cancer, prostate cancer, ovarian cancer, and cervical cancer.

In yet another embodiment of the present invention, the disease may be selected from the group consisting of hypertension, osteoporosis, irritable bowel syndrome, acute coronary syndrome, stroke, diabetes, atherosclerosis, obesity, peptic ulcer, Alzheimer's disease, emphysema, and skin diseases.

In another embodiment of the present invention, the vaccine may be modified and used for the purpose of increasing efficacy or reducing adverse effects. The modifications include modification of bacteria, modification using transformed bacteria, and modifications of extracellular vesicles that treat bacteria with chemicals, and these modifications are as described above.

In yet another embodiment of the present invention, the vaccine may be used by co-administering a drug or an immunoadjuvant for the purpose of increasing efficacy or reducing adverse effects, but is not limited thereto.

Method for Preparing Extracellular Vesicles with Reduced Toxicity

Yet another aspect of the present invention provides a method for preparing bacterial extracellular vesicles with reduced toxicity.

As an embodiment of the method for preparing extracellular vesicles with reduced toxicity, there is provided a method comprising the following steps: culturing bacteria in a chemically defined medium; and isolating bacterial extracellular vesicles secreted from the culture medium.

The bacterial extracellular vesicles with reduced toxicity prepared by the method may further comprise sterilizing the extracellular vesicles using a method selected from the group consisting of antibiotic treatment, UV exposure, gamma ray exposure, and filtering.

The method may further comprise isolating the extracellular vesicles having a size smaller than that of the bacteria and loaded with the drug.

The isolating step may be performed by using a method selected from the group consisting of ultracentrifugation, density gradient, filtration, dialysis, precipitation, chromatography, and free-flow electrophoresis.

In addition, the method of the present invention may further comprise removing the extracellular vesicles having a membrane with modified topology compared to the bacterial cell membrane. For example, by using an antibody that recognizes a cytoplasmic domain of the cell membrane protein, the extracellular vesicles with the cytoplasmic domain exposed to the outside may be removed.

Still another aspect of the present invention provides a method for reducing the toxicity of bacterial extracellular vesicles for delivering materials for treating and/or diagnosing diseases, wherein the method is characterized by comprising culturing the bacteria in a chemically defined medium.

Hereinafter, the present invention will be described in more detail with reference to Examples. These Examples are just illustrative of the present invention, and it will be apparent to those skilled in the art that it is not interpreted that the scope of the present invention is limited to these Examples.

### EXAMPLE 1

### Culture of E. coli with Reduced Toxicity of Lipopolysaccharides

In order to overcome the above-described problem, transformed E. coli was cultured using various chemically defined media with chemically defined components and mammalian cell culture media, and its growth was monitored.

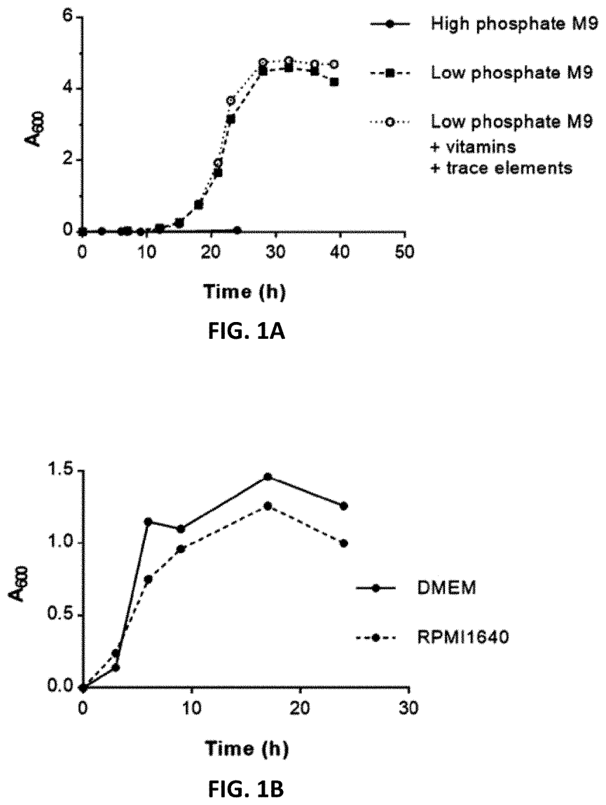

Specifically, as shown in the following [Table 1], a high concentration phosphate M9 (High Phosphate-M9) and low concentration phosphate M9 (Low Phosphate-M9) chemically defined medium, or a low concentration phosphate M9 chemically defined medium with vitamins and trace elements (M9+) were prepared. Using this, E. coli ΔmsbB prsA-EGF, which is E. coli ΔmsbB (the toxicity of lipopolysaccharides was reduced) transformed with pHCE-prsA-EGF vectors expressing a fusion protein of human EGF and bacterial inner membrane protein PrsA. The growth of the bacteria in each medium was confirmed by absorbance at wavelength 600 nm. As a result, the bacteria cannot be incubated in High Phosphate-M9 medium, but can be incubated in Low Phosphate-M9 medium, and their growth rate was not significantly different from that of culture in M9+ (FIG. 1A). In addition, E. coli ΔmsbB prsA-EGF can be incubated in DMEM and RPMI 1640, which are mammalian cell culture media, but the final cell concentration did not increase any more after the absorbance at wavelength 600 nm reached about 1 (FIG. 1B).

TABLE 1

| Medium | | |
|---|---|---|
| | High Phosphate-M9 | Low Phosphate-M9 |

| Component | Concentration | | | |
|---|---|---|---|---|
| Salts and glucose | | | | |
| Na₂HPO₄ | 90.2 | mM | 33.7 | mM |
| KH₂PO₄ | 22.0 | mM | 22.0 | mM |
| NH₄Cl | 18.7 | mM | 9.4 | mM |
| NaCl | 8.6 | mM | 8.6 | mM |
| MgSO₄ | 4.0 | mM | 1.0 | mM |
| CaCl₂ | 0.1 | mM | 0.3 | mM |

| Glucose | 0.4% | 0.4% | |
|---|---|---|---|
| Vitamins | | | |
| Thiamine | 0 | 4.0 | mM |
| Biotin | 0 | 4.0 | mM |

Trace elements

| | | | |
|---|---|---|---|
| EDTA | 0 | 13.4 | mM |
| FeCl₃•6H₂O | 0 | 3.1 | mM |
| ZnCl₂ | 0 | 0.62 | mM |
| CuCl₂•2H₂O | 0 | 0.076 | mM |
| CoCl₂•2H₂O | 0 | 0.042 | mM |
| H₃BO₃ | 0 | 0.162 | mM |
| MnCl₂•4H₂O | 0 | 0.008 | mM |

### EXAMPLE 2

### Culture of Various Bacteria

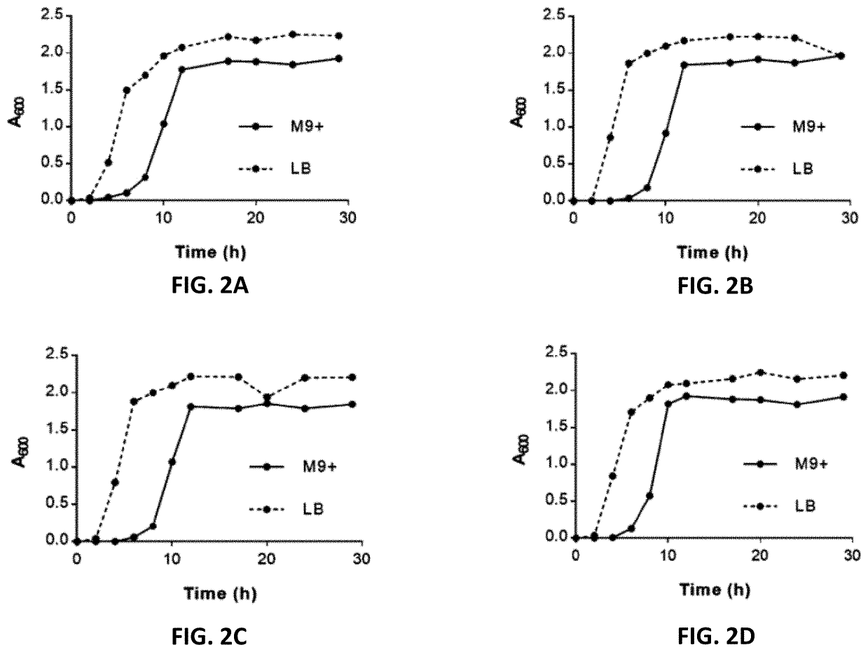

Various bacteria were cultured in M9+ and LB (a chemically undefined medium), and their growth were monitored. As a result of culturing E. coli ΔmsbB (FIG. 2A), which has reduced toxicity of lipopolysaccharides, S. aureus (FIG. 2B), S. enterica (FIG. 2c), B. subtilis (FIG. 2D) grew well in both M9+ and LB.

### EXAMPLE 3

### Isolation of Bacterial Extracellular Vesicles

To isolate bacterial extracellular vesicles, E. coli ΔmsbB prsA-EGF was cultured in LB, Low Phosphate-M9, M9+, and DMEM, respectively. In addition, in order to determine whether the expression of the prsA-EGF fusion protein differs in the production, composition and physiological activity of bacterial extracellular vesicles, E. coli ΔmsbB was cultured in LB, Low Phosphate-M9, and M9+. In addition, S. aureus was cultured in LB and M9+, and S. enterica as well as B. subtilis were cultured in M9+. Each culture medium was placed in high speed centrifuge tubes, and then centrifuged twice at 6,000×g for 20 minutes at 4° C. The supernatant from which bacteria were removed was filtered once through a membrane filter having a pore size of 0.45 μm, and then concentrated 50 times using a membrane capable of removing proteins having a molecular weight of 100 kDa or less. The concentrate was filtered once through a membrane filter having a pore size of 0.22 μm, and then placed in a 70 mL ultracentrifuge tube, followed by ultracentrifugation at 150,000×g for 3 hours at 4° C. The pellet was suspended in 2.5 mL of 50% OptiPrep, placed in a 5 mL ultracentrifuge tube, and 1.5 mL of 40% OptiPrep and 1.25 mL of 10% OptiPrep were sequentially placed thereon. Thereafter, ultracentrifugation was performed at 200,000×g for 2 hours at 4° C. Bacterial extracellular vesicles were obtained in a layer between 10% OptiPrep and 40% OptiPrep, filtered once through a membrane filter having a pore size of 0.22 μm, dispensed and stored at −80° C.

In the present specification, abbreviations shown in the following [Table 2] were used to systematically express various bacterial extracellular vesicles.

TABLE 2

| | | | Extracellular |
|---|---|---|---|
| Bacterial strain | Medium | Supplements* | vesicles |
| E. coli ΔmsbB | LB | − | ^{EGF}EV^{LB} |
| prsA-EGF | Low phosphate M9 | − | ^{EGF}EV^{M9} |
| | DMEM | + | ^{EGF}EV^{M9+} |
| | | − | ^{EGF}EV^{DMEM} |
| E. coli ΔmsbB | LB | − | EV^{LB} |
| | Low phosphate M9 | − | EV^{M9} |
| | | + | EV^{M9+} |
| S. aureus | LB | − | SA EV^{LB} |
| | Low phosphate M9 | + | SA EV^{M9+} |
| S. enterica | Low phosphate M9 | + | SE EV^{M9+} |
| B. subtilis | Low phosphate M9 | + | BS EV^{M9+} |

*vitamins and trace elements

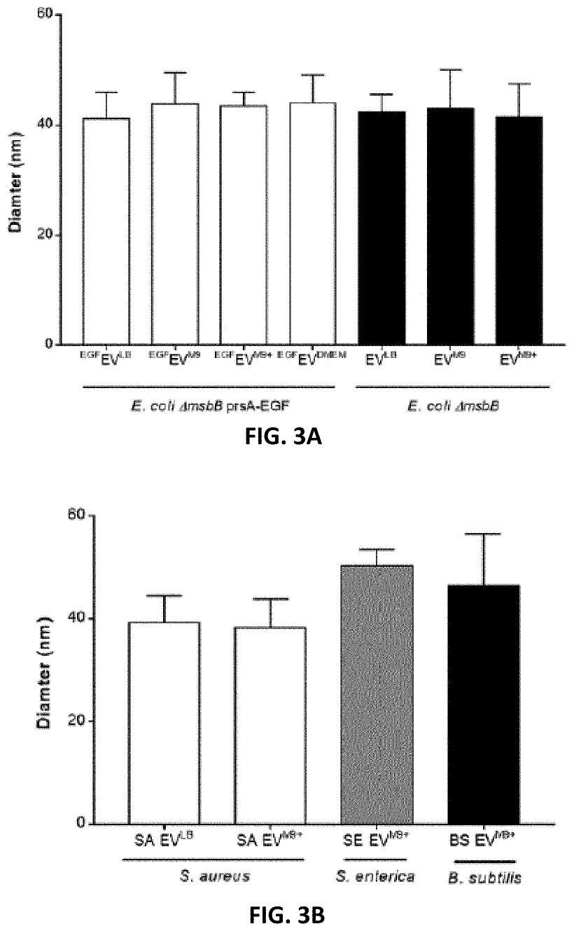

As a result of analyzing the size of the bacterial extracellular vesicles by a dynamic light scattering particle size analyzer, as shown in FIGS. 3A-B, the diameters of the bacterial extracellular vesicles derived under various conditions were similar, ranging 40-50 nm.

### EXAMPLE 4

### Quantification of Total Protein Amounts (Yield) and Numbers of Bacterial Extracellular Vesicles

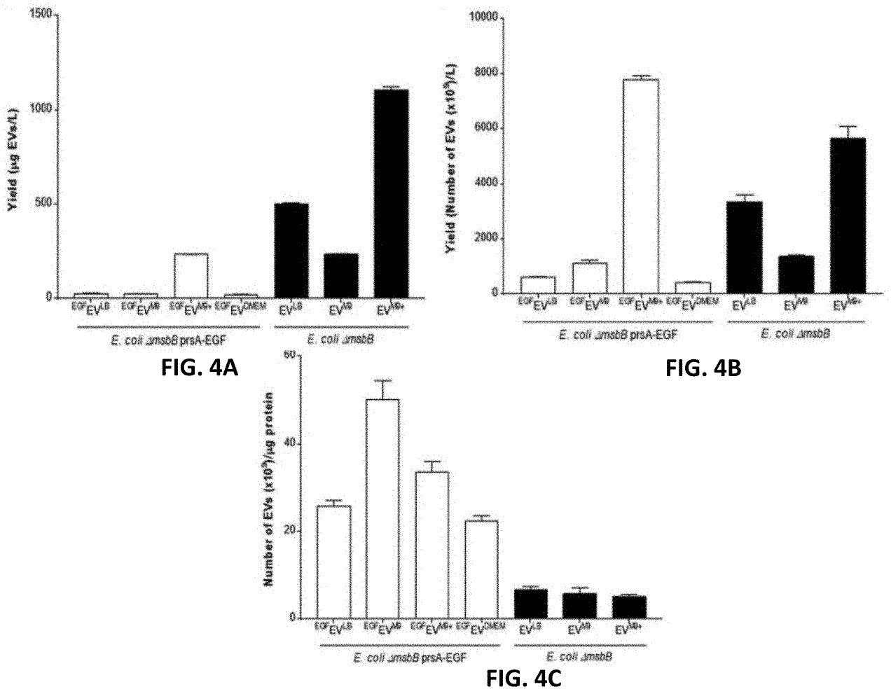

Total protein amounts (yield) of the bacterial extracellular vesicles obtained according to the method of Example 3 was quantified by Bradford protein assay, and numbers of the bacterial extracellular vesicles was quantified by nanoparticle tracking analysis. FIG. 4A is a graph measuring total protein amounts of bacterial extracellular vesicles derived from 1 liter culture medium, FIG. 4B is a graph measuring numbers of bacterial extracellular vesicles corresponding to 1 μg of protein, and FIG. 4C is a graph measuring numbers of bacterial extracellular vesicles derived from 1 liter culture medium. As shown in FIG. 4A, E. coli ΔmsbB secreted a greater amount of extracellular vesicles in terms of total protein amounts under all culture conditions than E. coli ΔmsbB prsA-EGF, but as shown in FIG. 4B, it was observed that E. coli ΔmsbB prsA-EGF secreted a significantly greater number of bacterial extracellular vesicles corresponding to 1 μg of protein than E. coli ΔmsbB. Therefore, as shown in FIG. 4C, depending on the culture conditions, E. coli ΔmsbB prsA-EGF secreted a greater number of extracellular vesicles in terms of the number of bacterial extracellular vesicles derived from 1 liter culture medium, or even in case of secreting a fewer number of extracellular vesicles, the ratio was significantly smaller than the differences in total protein amounts as compared to FIG. 4A.

### EXAMPLE 5

### Analysis of Protein Composition of Bacterial Extracellular Vesicles

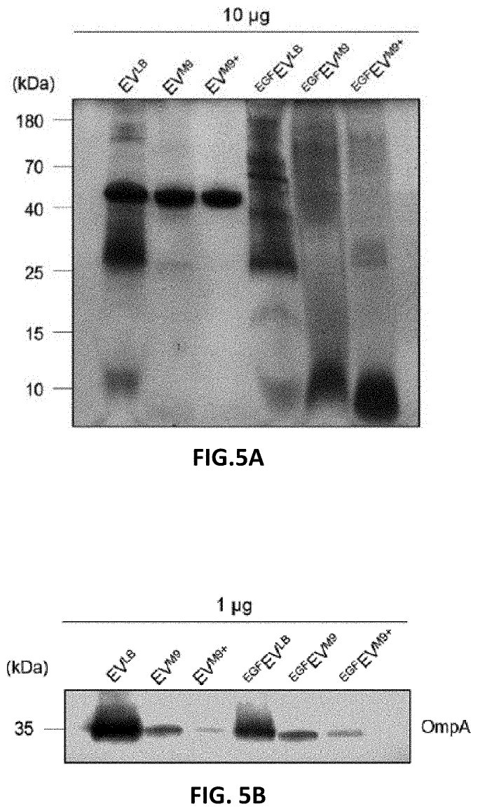

Bacterial extracellular vesicles derived from E. coli ΔmsbB and E. coli ΔmsbB prsA-EGF cultured in various media were similar in diameters, but in order to investigate the cause of the significant difference in the numbers of bacterial extracellular vesicles corresponding to 1 μg of protein, a total of 10 μg of EV^{LB}, EV^{M9}, EV^{M9+}, ^{EGF}Ev^{LB}, ^{EGF}EV^{M9}, and ^{EGF}EV^{M9+} were subjected to SDS-PAGE, and their protein compositions were shown to be significantly different (FIG. 5A). In addition, a total of 1 μg of EV^{LB}, EV^{M9}, EV^{M9+}, ^{EGF}Ev^{LB}, ^{EGF}EV^{M9}, and ^{EGF}EV^{M9+} were subjected to Western blotting against OmpA, an outer membrane protein, and various amounts of OmpA were present in various bacterial extracellular vesicles (FIG. 5B). Therefore, the amount of OmpA present in one extracellular vesicle as well as the types and amounts of various proteins were significantly different.

### EXAMPLE 6

### Anti-Tumor Activity of Gram-Negative Bacterial Extracellular Vesicles with Weakened Toxicity

Among the bacterial extracellular vesicles obtained according to the method of Example 3, the anti-tumor activities of ^{EGF}EV^{M9}, ^{EGF}EV^{DMEM}, and ^{EGF}EV^{LB}, as well as EV^{LB}, which is known to have anti-tumor activity, were verified in a mouse tumor model.

Specifically, a total of 28 heads of 5-week-old Balb/c mice (The Jackson Laboratory, Bar Harbor, Me.) were used in the experiment, and a total of 1×10⁶ cells of mouse colon cancer cells (CT26) were subcutaneously administered to the mice and grown. At 6 days after administration of the mouse colon cancer cells, the mice were divided into 7 experimental groups, each with 4 mice, and 100 μL of PBS solution containing ^{EGF}EV^{M9} (3 μL or 30 μL), or 100 μL of PBS solution containing ^{EGF}EV^{DMEM} (8 μL or 40 μL), 100 μL of PBS solution containing ^{EGF}EV^{LB} (6 μL), 100 μL of PBS solution containing EV^{LB} (10 μL), or 100 μL of PBS solution not containing extracellular vesicles as a control for each experimental group was administered into the tail vein twice a week (on the 6th, 10th, and 13th days of cell administration). The amount of extracellular vesicles administered to mice was to include an amount (3 μL of ^{EGF}EV^{M9} or 8 μL of ^{EGF}EV^{DMEM}) corresponding to the number of 5 μg of EV^{LB} (˜1×10¹⁰ EVs), 10 times (^{EGF}EV^{M9} 30 μL, ^{EGF}EV^{LB} 6 μL, EV^{LB} 10 μL), or 5 times (^{EGF}EV^{DMEM} 40 μL) of the amount of EV^{LB} to compare with the results of EV^{LB}.

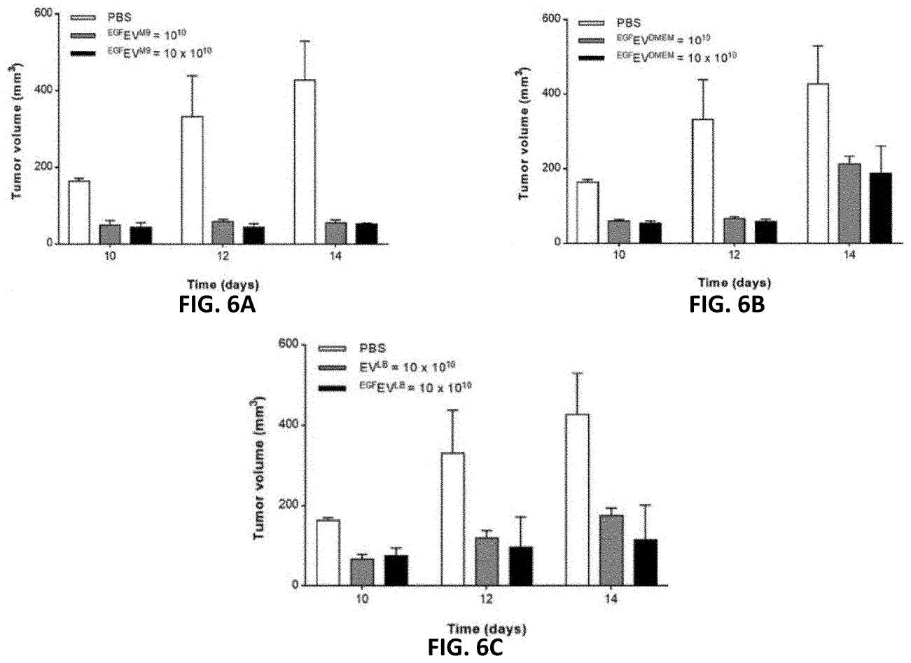

Next, for each experimental group, the size of the tumor tissue was measured at 10 days, 12 days, and 14 days after the administration of colon cancer cells. The volume (V) of the tumor tissue was calculated by measuring the longest length (l) and the perpendicular length (s) and using the formula of V=l×s²/2. FIGS. 6A-C show the results of measuring the size of the tumor tissue after subcutaneous administration of the colon cancer cells. Compared to the control group administered only with PBS, the sizes of tumor tissues continuously decreased in the group administered with ^{EGF}EV^{M9}, regardless of EV doses (FIG. 6A). In the group administered with ^{EGF}EV^{DMEM}, the sizes of tumor tissues decreased in a dose-dependent manner (FIG. 6B). The sizes of tumor tissues also decreased in the group administered with EV^{LB} or ^{EGF}EV^{LB}, when compared to the control group administered only with PBS (FIG. 6C).

In addition, the changes in body weight, body temperature, movement, and eye exudates or piloerection of the mice in each experimental group were continuously observed, and whether the appearance of the tumor tissues turned black was observed. As a result, compared to the control group administered only with PBS, in the group administered with ^{EGF}EV^{M9} and ^{EGF}EV^{DMEM}, the tumor tissues did not get blackened. In addition, there were no significant changes in body weight, body temperature, and movement of the mice. Adverse effects, such as eye exudates and piloerection, were not observed, and no mice were dead. However, administration of EV^{LB} or ^{EGF}EV^{LB} effectively inhibited tumor growth, but at the same time, the tumor tissues got blackened in these groups. In addition, various adverse effects including body weight loss, decrease in movement, eye exudates, piloerection, and even death were observed.

Taken together, ^{EGF}EV^{M9} and ^{EGF}EV^{DMEM} not only had similar or better in anti-tumor effects when compared to EV^{LB}, but also did not induce adverse effects even when administered at least 10 times of the amounts which could induce anti-tumor effects.

### EXAMPLE 7

### Anti-Tumor Activity of Gram-Positive Bacterial Extracellular Vesicles with Reduced Toxicity

Among the bacterial extracellular vesicles obtained according to the method of Example 3, anti-tumor activity of SA EV^{M9+} was verified in a mouse tumor model.

Specifically, a total of 12 heads of 5-week-old Balb/c mice were used in the experiment, and a total of 1×10⁶ cells of mouse colon cancer cells (CT26) were subcutaneously administered to the mice and grown. At 6 days after administration of the mouse colon cancer cells, the mice were divided into 3 experimental groups, each with 4 mice, and 100 μL of PBS solution containing SA EV^{M9+} (1 μL or 10 μL), or 100 μL of PBS solution containing no extracellular vesicles as a control was administered into the tail vein twice a week (on the 6th, 10th, and 13th days of cell administration). To compare with the results of EV^{LB}, the amount of extracellular vesicles administered to mice was to include an amount corresponding to the number of 5 μg of EV^{LB} (˜1×10¹⁰ EVs) (SA EV^{M9+} 1 μL) or 10 times thereof (SA EV^{M9+} 10 μL).

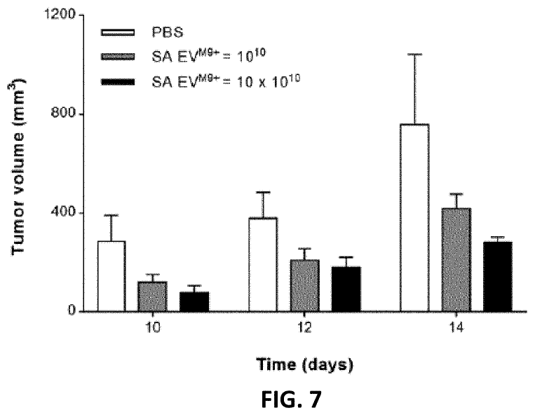

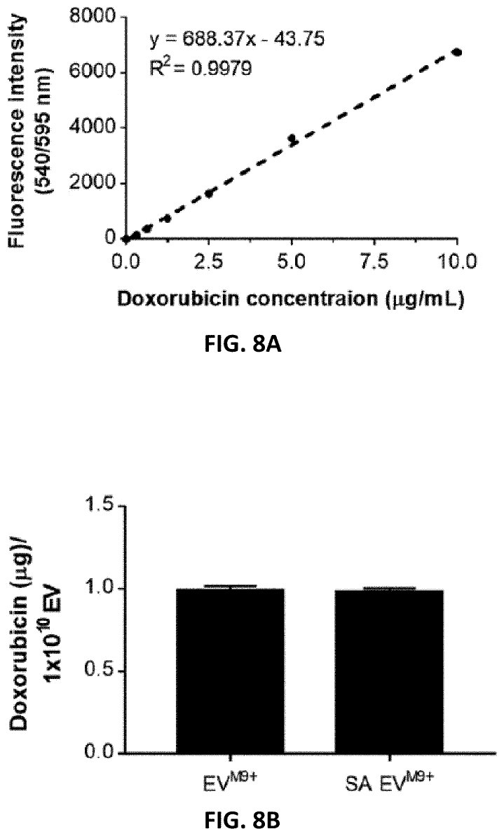

Next, for each experimental group, the size of the tumor tissue was measured at 10 days, 12 days, and 14 days after the administration of colon cancer cells. The volume (V) of the colon cancer tissue was calculated by measuring the longest length (1) and the perpendicular length (s) and using the formula of V=l×s²/2. FIG. 7 shows the results of measuring the size of the tumor tissue after subcutaneous administration of the colon cancer cells. Compared to the control group administered only with PBS, the sizes of tumor tissues continuously decreased in the group administered with SA EV^{M9+}, in a dose-dependent manner.

In addition, the changes in body weight, body temperature, movement, and eye exudates or piloerection in each experimental group were continuously observed, and whether the appearance of the tumor tissues turned black was observed. As a result, compared to the control group administered only with PBS, in the group administered with SA EV^{M9+}, the tumor tissues did not get blackened. In addition, there were no significant changes in body weight, body temperature, and movement of the mice. Adverse effects, such as eye exudates and piloerection, were not observed, and no mice were dead.

Taken together, SA EV^{M9+} not only had similar or better in anti-tumor effects when compared to EV^{LB}, but also did not induce adverse effects even when administered at least 10 times of the amounts which could induce anti-tumor effects.

### EXAMPLE 8

### Drug Delivery Using Bacterial Extracellular Vesicles with Reduced Toxicity

Among the bacterial extracellular vesicles obtained according to the method of Example 3, the following experiment was conducted to confirm whether drugs can be delivered using EV^{M9+} and SA EV^{M9+}. In this experiment, doxorubicin was used as an example of a drug.

EV^{M9+} and SA EV^{M9+} obtained according to the method of Example 3 were mixed with doxorubicin at a concentration of 0.8 mg/mL in a 1:1 ratio for 12 hours at 4° C. Then, ultracentrifugation was performed at 150,000×g for 3 hours at 4° C. to separate bacterial extracellular vesicles loaded with doxorubicin and free doxorubicin in the solution. Doxorubicin-loaded bacterial extracellular vesicles were subjected to fluorescence measurement and nanoparticle tracking analysis. Both doxorubicin-loaded EV^{M9+} (^{Dox}EV^{M9+}) and SA EV^{M9+} (SA ^{Dox}EV^{M9+}) contained 1 μg of doxorubicin in 1×10¹⁰ EVs.

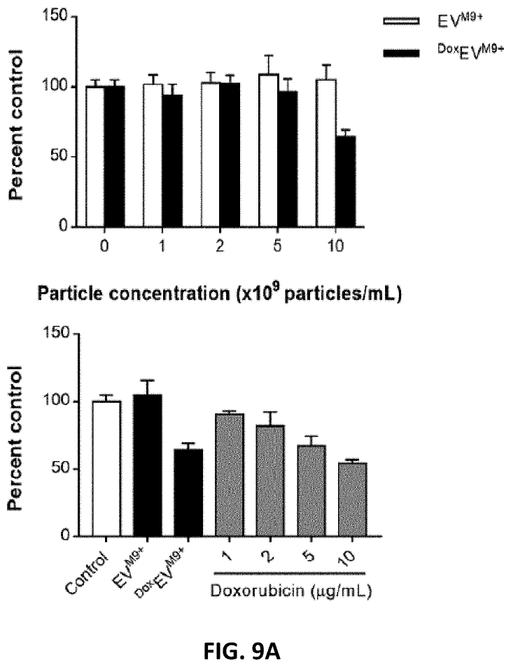

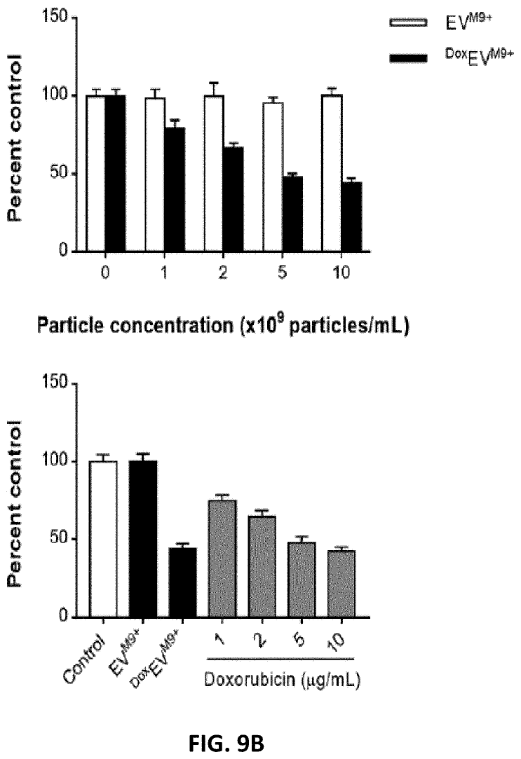

Mouse colon cancer cells (CT26, 2×10³ cells/well) and human vascular endothelial cells (HMEC-1, 4×10³ cells/well) were cultured in a 96-well plate for one day. PBS (Control) and various amounts of EV^{M9+} as well as ^{Dox}EV^{M9+} were treated to these cells for 24 hours. The survival rate of these cells were measured with WST-1 assay, and the results are shown in FIGS. 9A-B.

As can be seen in FIGS. 9A-B, EV^{M9+} did not affect the death of cancer cells and vascular endothelial cells, regardless of its concentration, but ^{Dox}EV^{M9+} induced the death of these cells in a dose-dependent manner. While ^{Dox}EV^{M9+} 1×10¹⁰ EVs/mL is loaded with 1 μg/mL of doxorubicin, ^{Dox}EV^{M9+} showed greater effects in inducing cell death, when compared with the same concentration of free doxorubicin, and the effects were equivalent to 10 times higher concentration of free doxorubicin.

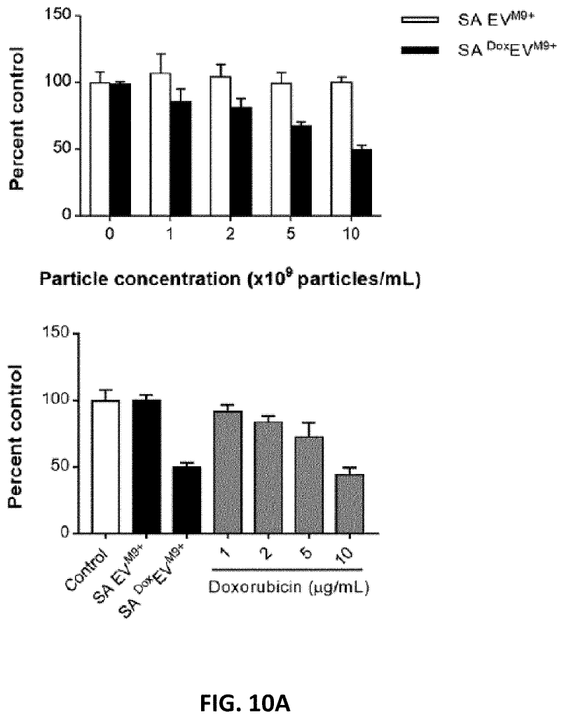

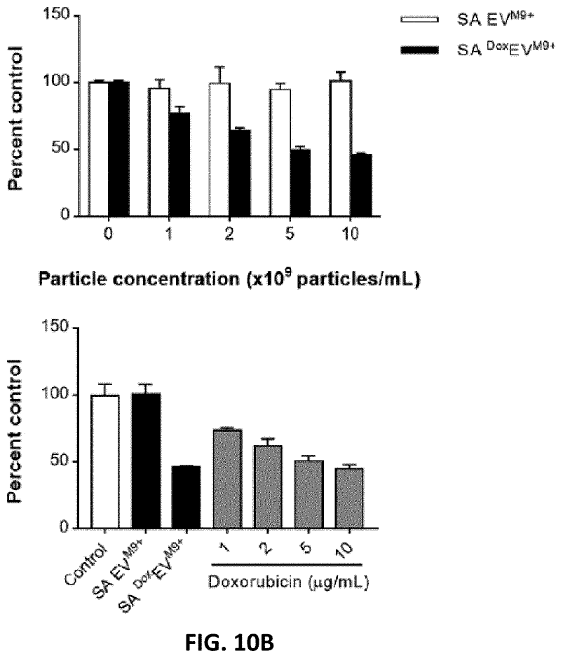

Meanwhile, SA EV^{M9+} and SA ^{Dox}EV^{M9+} were also treated to mouse colorectal cancer cells and human vascular endothelial cells in the same manner as described above to confirm the survival rate of the cells. As can be seen in FIGS. 10A-B, SA EV^{M9+} did not affect the death of cancer cells and vascular endothelial cells, regardless of its concentration, but SA ^{Dox}EV^{M9+} induced the death of these cells in a dose-dependent manner While SA ^{Dox}EV^{M9+} 1×10¹⁰ EVs/mL is loaded with 1 μg/mL of doxorubicin, SA ^{Dox}EV^{M9+} showed greater effects in inducing cell death, when compared with the same concentration of free doxorubicin, and the effects were equivalent to 10 times higher concentration of free doxorubicin.

Taken together, drugs can be delivered more efficiently using bacterial extracellular vesicles loaded with the drugs, and therapeutic effects can be enhanced by delivering the drugs through the extracellular vesicles when the same amount of the drugs are administered.

### EXAMPLE 9

### Vaccine Using Bacterial Extracellular Vesicles with Reduced Toxicity

Among the bacterial extracellular vesicles obtained according to the method of Example 3, EV^{M9+} and SA EV^{M9+} were assessed if they could be utilized as vaccines using a mouse model.

Specifically, a total of 15 heads of 5-week-old C57BL/6 (The Jackson Laboratory) were used in the experiment, and the mice were divided into 5 experimental groups, each with 3 mice. A total of 100 μL of PBS solution containing EV^{M9+} (0.5 μL or 2.5 μL), 100 μL of PBS solution containing SA EV^{M9+} (7 μL or 33 μL), or 100 μL of PBS solution containing no extracellular vesicles as a control was administered into the thigh muscles twice with a week interval. The amount of extracellular vesicles administered to mice is equivalent to 1 μg (EV^{M9+} 0.5 μL or SA EV^{M9+} 7 μL) or to 5 μg (EV^{M9+} 2.5 μL or SA EV^{M9}+33 μL).

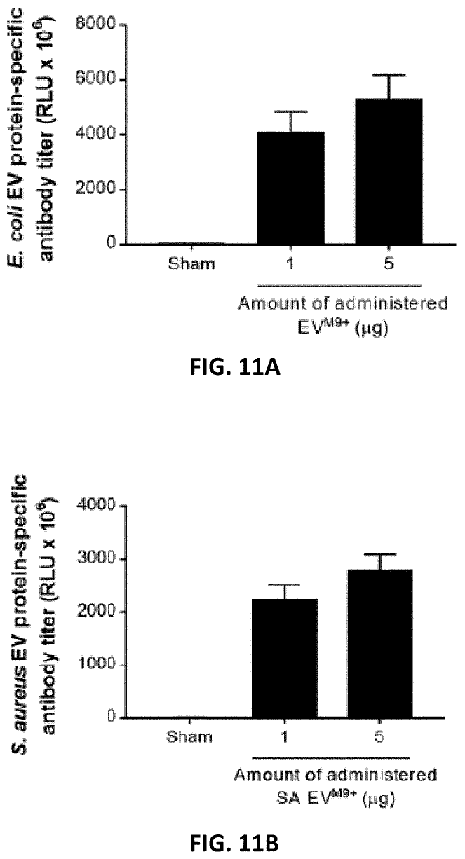

One week after the last administration of extracellular vesicles into the mice, some blood was obtained by eye bleeding, and the extracellular vesicle-specific antibodies present in the blood were measured. After coating a black 96-well plate with EV^{M9+} or SA EV^{M9+} 200 ng/well, the mouse sera diluted 1:500 with 1% BSA/PBS were added for 2 hours at room temperature. Then, peroxidase-conjugated antibodies against mouse antibodies were added to be observed with enzyme-linked immunesorbent assay (ELISA). FIGS. 11A-B are a result of observing the amount of antibody specific to EV^{M9+} (FIG. 11A) or SA EV^{M9+} (FIG. 11B) in mouse serum, and shows that the formation of specific antibodies to the extracellular vesicles increased in a dose-dependent manner. This means that when the extracellular vesicles are administered into the muscle twice or more, specific antibodies against proteins contained in the bacterial extracellular vesicles are formed.

Taken together, the differences in toxicity as well as safety and efficacy of extracellular vesicles obtained from various conditions (various Gram-negative and Gram-positive bacteria, transformed strains thereof, and culture media) as therapeutic agents, drug delivery systems, and/or vaccine delivery systems can be investigated, and various components (proteins, nucleic acids, lipids, peptidoglycans, etc.) of these extracellular vesicles can be compared and analyzed using multi-omics technology. The substances involved in toxicity and efficacy thereby can be elucidated, and novel extracellular vesicles or substances with reduced adverse effects and enhanced efficacy as therapeutic agents, drug delivery systems, or vaccine delivery systems can be developed.

Bacterial extracellular vesicles with reduced toxicity according to the present invention can be additionally 1) subjected to regulated in sizes and homogenized (size modulation), or 2) reduced in adverse effects, increased in stability in vitro and in vivo including the bloodstream, improved in efficacy as therapeutic agents, drug delivery systems, and/or vaccine delivery systems, or loaded or displayed with compounds, peptides, proteins, fusion proteins, nucleic acids, aptamers, toxins, various types of antigens, polymers, lipids, and complexes thereof, alone or combination; and 3) by combining the above methods, the adverse effects of bacterial extracellular vesicles can be reduced, thereby effectively enhancing the stability and efficacy as therapeutic agents, drug delivery systems, and/or vaccine delivery systems.

In addition, bacterial extracellular vesicles of the present invention, which is loaded with a substance for disease treatment or vaccine, and its preparation method, can be used for treatment, drug delivery, vaccines, or experiments in vitro or in vivo.

What is claimed is:

1. A pharmaceutical composition for treating or diagnosing diseases, comprising bacterial extracellular vesicles with reduced toxicity, wherein the bacteria are cultured in a chemically defined medium.

2. The pharmaceutical composition of claim 1, wherein the bacteria are transformed bacteria.

3. The pharmaceutical composition of claim 2, wherein the bacteria are bacteria transformed to reduce the toxicity of extracellular vesicles.

4. The pharmaceutical composition of claim 2, wherein the bacteria are bacteria having at least one genotype selected from the group consisting of ΔmsbB (ΔlpxM), ΔlpxA, ΔlpxB, ΔlpxC, ΔlpxD, ΔlpxH, ΔlpxK, ΔlpxL, and ΔwaaA.

5. The pharmaceutical composition of claim 2, wherein the bacteria are bacteria transformed to express a cell membrane fusion material.

6. The pharmaceutical composition of claim 1, wherein the chemically defined medium includes one or more carbon sources, one or more nitrogen sources, and one or more inorganic salts.

7. The pharmaceutical composition of claim 1, wherein the chemically defined medium is selected from the group consisting of M9 Minimal Medium, Dulbecco's Modified Eagle Medium (DMEM), Roswell Park Memorial Institute 1640 (RPMI 1640) Medium, Minimum Essential Medium (MEM), MEMα, Opti-MEM, Iscove's Modified Dulbecco's Medium (IMDM), DMEM/Nutrient Mixture F-12 (DMEM/F-12) Medium, McCoy's 5A Medium, Medium 199, Leibovitz's L-15 Medium, Connaught Medical Research Laboratories (CMRL) Medium, Ham's F-12K Medium, BGJb Medium, William's E Medium, Basal Medium Eagle(BME), Glasgow's MEM (GMEM), Brinster's Medium for Ovum Culture (BMOC), Fischer's Medium and MCDB 131 Medium.

8. The pharmaceutical composition of claim 1, wherein the disease is selected from the group consisting of thyroid cancer, hepatic cancer, osteosarcoma, oral cancer, brain tumor, gall bladder cancer, colon cancer, lymphoma, bladder cancer, leukemia, small intestine cancer, tongue cancer, esophageal cancer, renal cancer, gastric cancer, breast cancer, pancreatic cancer, lung cancer, skin cancer, testicular cancer, penile cancer, prostate cancer, ovarian cancer, and cervical cancer.

9. The pharmaceutical composition of claim 1, wherein the disease is selected from the group consisting of hypertension, osteoporosis, irritable bowel syndrome, acute coronary syndrome, stroke, diabetes, atherosclerosis, obesity, peptic ulcer, Alzheimer's disease, emphysema, skin disease, skin infection, respiratory infection, urogenital infection, bone joint infection, central nervous system infection, and sepsis.

10. The pharmaceutical composition of claim 8, wherein the composition further comprises a drug of enhancing an anti-cancer effect.

11. The pharmaceutical composition of claim 10, wherein the drug is loaded into the bacterial extracellular vesicles.

12. The pharmaceutical composition of claim 1, wherein the bacterial extracellular vesicles have reduced toxicity compared to bacterial extracellular vesicles cultured in lysogeny broth (LB).

13. A composition for delivering materials for treating or diagnosing diseases comprising bacterial extracellular vesicles with reduced toxicity, loaded with a material for treating or diagnosing diseases, wherein the bacteria are cultured in a chemically defined medium.

14. The composition for delivering materials of claim 13, wherein the material for treatment or diagnosis is selected from the group consisting of anti-cancer agents, anti-inflammatory agents, angiogenesis inhibitors, peptides, proteins, vaccines, toxins, nucleic acids, beads, microparticles, nanoparticles, fluorescent proteins, and quantum dots.

15. A vaccine composition for preventing or treating diseases comprising bacteria-derived extracellular vesicles with reduced toxicity, wherein the bacteria are cultured in a chemically defined medium, and the bacteria are bacteria transformed to express a fusion protein of a membrane protein of the extracellular vesicles and an antigen; or bacteria transformed to express a fusion protein of a luminal cargo of the extracellular vesicles and an antigen.

16. The vaccine composition of claim 15, wherein the disease is an infection caused by bacteria, viruses or fungi.

17. The vaccine composition of claim 15, wherein the disease is selected from the group consisting of thyroid cancer, hepatic cancer, osteosarcoma, oral cancer, brain tumor, gall bladder cancer, colon cancer, lymphoma, bladder cancer, leukemia, small intestine cancer, tongue cancer, esophageal cancer, renal cancer, gastric cancer, breast cancer, pancreatic cancer, lung cancer, skin cancer, testicular cancer, penile cancer, prostate cancer, ovarian cancer, and cervical cancer, or selected from the group consisting of hypertension, osteoporosis, irritable bowel syndrome, acute coronary syndrome, stroke, diabetes, atherosclerosis, obesity, peptic ulcer, Alzheimer's disease, emphysema, and skin diseases.

18. The vaccine composition of claim 15, wherein an antigen protein or a peptide is loaded into the bacterial extracellular vesicles.

19. A method for preparing bacterial extracellular vesicles with reduced toxicity, comprising (a) culturing bacteria in a chemically defined medium; and (b) isolating the bacterial extracellular vesicles secreted from the culture medium.

20. A method for reducing toxicity of bacterial extracellular vesicles for delivering materials, comprising: (a) culturing bacteria in a chemically defined medium; (b) isolating bacterial extracellular vesicles secreted from the culture medium; (c) adding and culturing a material for treating or diagnosing diseases to a suspension containing the isolated bacterial extracellular vesicles; and (d) isolating the bacterial extracellular vesicles loaded with the material for treating or diagnosing diseases secreted from the culture medium.
